(19) United States
(12) Patent Application Publication
(10) Pub. No.: US 2009/0215054 A1
(43) Pub. Date: Aug. 27, 2009

(54) PROGNOSIS INDICATORS FOR SOLID HUMAN TUMORS

(76) Inventors: Scott L. Carter, Cambridge, MA (US); Zoltan Szallasi, Boston, MA (US); Aron Eklund, Copenhagen (DK)

Correspondence Address: K&L Gates LLP, STATE STREET FINANCIAL CENTER, One Lincoln Street, BOSTON, MA, 02111-2950, US

(21) Appl. No.: 12/097,175

(22) PCT Filed: Dec. 13, 2006
(86) PCT No.: PCT/US2006/047662

§ 371 Date: Mar. 20, 2009

Related U.S. Application Data
(60) Provisional application No. 60/749,754, filed on Dec. 13, 2005.

(51) Int. Cl. C12Q 1/68; C07H 21/04

(52) U.S. Cl. 435/6; 536/23.1

(57) ABSTRACT

The present teachings provide methods for predicting the clinical outcome of the treatment of human solid tumors. In some embodiments, the method includes measuring in the cells of a tumor the expression level of a set of genes whose change is related to chromosomal instability; taking a statistical measure of the expression level of the set of measured genes; and if the statistical measure of the expression level of the set of measured genes is elevated, determining that the prognosis is poor. Another aspect of the present teachings is the sets of genes, which are useful in predicting the outcome of treatment of solid tumors.

### GOVERNMENT SUPPORT

The United States government has certain rights to this invention pursuant to Grant No. 1PO1CA 092644-01 from the National Cancer Institute.

### FIELD

The present teachings relate generally to the field of cancer diagnostics and treatment, and more specifically to the determination of the likelihood that the outcome of a treatment will be successful.

### BACKGROUND

The treatment options for solid human tumors are multifold. Solid tumors can be treated chemotherapeutically, radiologically, surgically, or with a combination of these therapies. Each therapy produces undesirable side effects, which may be extensive enough that some patients cannot complete the course of treatment. The side effects of cancer therapy also have a severe impact on the quality of life of these patients.

As a result, if the clinician can determine prior to treatment how refractory the tumor will respond to treatment, an appropriate treatment can be selected having the least side effects for the patient. The present teachings provide a method for determining likelihood of clinical outcome based on the malignancy of the tumor.

### SUMMARY

The present teachings relate to methods for predicting the outcome of the treatment of solid human tumors. In various embodiments, the methods generally include measuring in a particular solid tumor cancer type the degree of chromosomal abnormalities and/or the expression levels of a large number of genes; identifying a subset of the measured genes characteristic of chromosomal instability (CIN); and determining in clinical samples whether the CIN signature accurately predicts the outcome of the treatment of the solid tumor. The methods can include the use of the CIN signature to analyze a tumor of a patient to determine the prognosis of the cancer and whether treatment is likely to be successful.

In certain embodiments, the method comprises measuring in solid tumor cells the mRNA expression of at least 25 genes in the following set of genes:

| | |
|---|---|
| 1 | TPX2 |
| 2 | PRC1 |
| 3 | FOXM1 |
| 4 | CDC2 |
| 5 | C20orf24/TGIF2 |
| 6 | MCM2 |
| 7 | H2AFZ |
| 8 | TOP2A |
| 9 | PCNA |
| 10 | UBE2C |
| 11 | MELK |
| 12 | TRIP13 |
| 13 | CNAP1 |
| 14 | MCM7 |
| 15 | RNASEH2A |
| 16 | RAD51AP1 |
| 17 | KIF20A |
| 18 | CDC45L |
| 19 | MAD2L1 |
| 20 | ESPL1 |
| 21 | CCNB2 |
| 22 | FEN1 |
| 23 | TTK |
| 24 | CCT5 |
| 25 | RFC4 |
| 26 | ATAD2 |
| 27 | ch-TOG |
| 28 | NUP205 |
| 29 | CDC20 |
| 30 | CKS2 |
| 31 | RRM2 |
| 32 | ELAVL1 |
| 33 | CCNB1 |
| 34 | RRM1 |
| 35 | AURKB |
| 36 | MSH6 |
| 37 | EZH2 |
| 38 | CTPS |
| 39 | DKC1 |
| 40 | OIP5 |
| 41 | CDCA8 |
| 42 | PTTG1 |
| 43 | C10 or f3 |
| 44 | H2AFX |
| 45 | CMAS |
| 46 | BRRN1 |
| 47 | MCM10 |
| 48 | LSM4 |
| 49 | MTB |
| 50 | ASF1B |
| 51 | ZWINT |
| 52 | TOPK |
| 53 | FLJ10036 |
| 54 | CDCA3 |
| 55 | ECT2 |
| 56 | CDC6 |
| 57 | UNG |
| 58 | MTCH2 |
| 59 | RAD21 |
| 60 | ACTL6A |
| 61 | GPI/MGC13096 |
| 62 | SFRS2 |
| 63 | HDGF |
| 64 | NXT1 |
| 65 | NEK2 |
| 66 | DHCR7 |
| 67 | STK6 |
| 68 | NDUFAB1 |
| 69 | KIAA0286 |
| 70 | KIF4A |
| 71 | SNRPB/GC10715 |
| 72 | UCK2 |
| 73 | PARP1 |
| 74 | RAD54L |
| 75 | NUSAP1 |
| 76 | RFC5 |
| 77 | TK1 |
| 78 | WBP11 |
| 79 | SYNCRIP/SNX14 |
| 80 | BIRC5/AFMID |
| 81 | HNRPAB |
| 82 | TACC3 |
| 83 | MKI67 |
| 84 | CENPF |
| 85 | Spc25 |
| 86 | C20 or f172 |
| 87 | PTBP1 |
| 88 | DLG7 |
| 89 | POLR2K |
| 90 | IARS |
| 91 | HPRT1 |
| 92 | NSDHL |
| 93 | KNTC2 |
| 94 | RAMP |
| 95 | C10 or f7 |
| 96 | C12 or f14 |
| 97 | SNRPD1 |
| 98 | FLJ20989 |
| 99 | NIF3L1 |
| 100 | DER1 |

taking a statistical measure of the expression level of the measured genes; and if the statistical measure of the expression level of the measured genes is elevated, determining, to a 99% confidence level, that the prognosis is poor. It should be understood that the other gene sets described herein are equally applicable to the above described method.

In certain embodiments, the solid tumor is of a cancer selected from lung cancer, prostate cancer, medulloblastoma, glioma, breast cancer, and lymphoma.

In some embodiments, the statistical measure of the expression level of the measured genes is a linear combination of the expression level of the genes in the set of genes. In particular embodiments, the linear combination of the expression level in the set of genes is a combination of weighted expression levels. In various embodiments, the linear combination of the expression level in the set of genes is the mean of the logarithm of each of the expression levels. In certain embodiments, the statistical measure of the expression level of the measured genes is elevated relative to the expression level of the measured genes from a tumor whose prognosis is good.

In some embodiments, the present teachings relate to a method for predicting outcome of the treatment of the human solid tumors. In these embodiments, the method generally includes the steps of measuring in the cells of a tumor the expression level of a set of genes (or subset of a gene set) whose change is related to chromosomal instability; taking a statistical measure of the expression level of the set of measured genes; and if the statistical measure of the expression level of the set of measured genes is elevated, determining that the prognosis is poor. In various embodiments, chromosomal instability can be measured by array comparative genomic hybridization (aCGH) and/or counting the number of morphologically visible chromosomal aberrations by the application of chromosome visualization methods such as spectral karyotyping (SKY). Such techniques can be used in conjunction with expression levels or to correlate and/or corroborate expression levels.

Another aspect of the present teachings is a set of genes or data from a set of genes, e.g., expression level data, useful in determining the outcome of treatment of solid tumors. In some embodiments, the set of genes comprises or consists essentially of:

| | |
|---|---|
| 1 | TPX2 |
| 2 | PRC1 |
| 3 | FOXM1 |
| 4 | CDC2 |
| 5 | C20 or f24/TGIF2 |
| 6 | MCM2 |
| 7 | H2AFZ |
| 8 | TOP2A |
| 9 | PCNA |
| 10 | UBE2C |
| 11 | MELK |
| 12 | TRIP13 |
| 13 | CNAP1 |
| 14 | MCM7 |
| 15 | RNASEH2A |
| 16 | RAD51AP1 |
| 17 | KIF20A |
| 18 | CDC45L |
| 19 | MAD2L1 |
| 20 | ESPL1 |
| 21 | CCNB2 |
| 22 | FEN1 |
| 23 | TTK |
| 24 | CCT5 |
| 25 | RFC4 |
| 26 | ATAD2 |
| 27 | ch-TOG |
| 28 | NUP205 |
| 29 | CDC20 |
| 30 | CKS2 |
| 31 | RRM2 |
| 32 | ELAVL1 |
| 33 | CCNB1 |
| 34 | RRM1 |
| 35 | AURKB |
| 36 | MSH6 |
| 37 | EZH2 |
| 38 | CTPS |
| 39 | DKC1 |
| 40 | OIP5 |
| 41 | CDCA8 |
| 42 | PTTG1 |
| 43 | C10orf3/CEP55 |
| 44 | H2AFX |
| 45 | CMAS |
| 46 | BRRN1 |
| 47 | MCM10 |
| 48 | LSM4 |
| 49 | MTB |
| 50 | ASF1B |
| 51 | ZWINT |
| 52 | TOPK |
| 53 | FLJ10036 |
| 54 | CDCA3 |
| 55 | ECT2 |
| 56 | CDC6 |
| 57 | UNG |
| 58 | MTCH2 |
| 59 | RAD21 |
| 60 | ACTL6A |
| 61 | GPI and MGC13096 |
| 62 | SFRS2 |
| 63 | HDGF |
| 64 | NXT1 |
| 65 | NEK2 |
| 66 | DHCR7 |
| 67 | STK6 |
| 68 | NDUFAB1 |
| 69 | KIAA0286 |
| 70 | KIF4A |
| 71 | SNRPB/MGC10715 |
| 72 | UCK2 |
| 73 | PARP1 |
| 74 | RAD54L |
| 75 | NUSAP1 |
| 76 | RFC5 |
| 77 | TK1 |
| 78 | WBP11 |
| 79 | SYNCRIP/SNX14 |
| 80 | BIRC5 and AFMID |
| 81 | HNRPAB |
| 82 | TACC3 |
| 83 | MKI67 |
| 84 | CENPF |
| 85 | Spc25 |
| 86 | C20 or f172 |
| 87 | PTBP1 |
| 88 | DLG7 |
| 89 | POLR2K |
| 90 | IARS |
| 91 | HPRT1 |
| 92 | NSDHL |
| 93 | KNTC2 |
| 94 | RAMP |
| 95 | C10 or f7 |
| 96 | C12 or f14 |
| 97 | SNRPD1 |
| 98 | FLJ20989 |
| 99 | NIF3L1 |
| 100 | DER1 |

or

| | |
|---|---|
| 1 | TPX2 |
| 2 | PRC1 |
| 3 | FOXM1 |
| 4 | CDC2 |
| 5 | C20 or f24/TGIF2 |
| 6 | MCM2 |
| 7 | H2AFZ |
| 8 | TOP2A |
| 9 | PCNA |
| 10 | UBE2C |
| 11 | MELK |
| 12 | TRIP13 |
| 13 | CNAP1 |
| 14 | MCM7 |
| 15 | RNASEH2A |
| 16 | RAD51AP1 |
| 17 | KIF20A |
| 18 | CDC45L |
| 19 | MAD2L1 |
| 20 | ESPL1 |
| 21 | CCNB2 |
| 22 | FEN1 |
| 23 | TTK |
| 24 | CCT5 |
| 25 | RFC4 |
| 26 | ATAD2 |
| 27 | ch-TOG |
| 28 | NUP205 |
| 29 | CDC20 |
| 30 | CKS2 |
| 31 | RRM2 |
| 32 | ELAVL1 |
| 33 | CCNB1 |
| 34 | RRM1 |
| 35 | AURKB |
| 36 | MSH6 |
| 37 | EZH2 |
| 38 | CTPS |
| 39 | DKC1 |
| 40 | OIP5 |
| 41 | CDCA8 |
| 42 | PTTG1 |
| 43 | CEP55/C10orf3 |
| 44 | H2AFX |
| 45 | CMAS |
| 46 | BRRN1 |
| 47 | MCM10 |
| 48 | LSM4 |
| 49 | MTB |
| 50 | ASF1B |
| 51 | ZWINT |
| 52 | TOPK |
| 53 | FLJ10036 |
| 54 | CDCA3 |
| 55 | ECT2 |
| 56 | CDC6 |
| 57 | UNG |
| 58 | MTCH2 |
| 59 | RAD21 |
| 60 | ACTL6A |
| 61 | GPI/MGC13096 |
| 62 | SFRS2 |
| 63 | HDGF |
| 64 | NXT1 |
| 65 | NEK2 |
| 66 | DHCR7 |
| 67 | STK6 |
| 68 | NDUFAB1 |
| 69 | KIAA0286 |
| 70 | KIF4A |

or

| | |
|---|---|
| 1 | TPX2 |
| 2 | PRC1 |
| 3 | FOXM1 |
| 4 | CDC2 |
| 5 | C20 or f24/TGIF2 |
| 6 | MCM2 |
| 7 | H2AFZ |
| 8 | TOP2A |
| 9 | PCNA |
| 10 | UBE2C |
| 11 | MELK |
| 12 | TRIP13 |
| 13 | CNAP1 |
| 14 | MCM7 |
| 15 | RNASEH2A |
| 16 | RAD51AP1 |
| 17 | KIF20A |
| 18 | CDC45L |
| 19 | MAD2L1 |
| 20 | ESPL1 |
| 21 | CCNB2 |
| 22 | FEN1 |
| 23 | TTK |
| 24 | CCT5 |
| 25 | RFC4 |

or

| | |
|---|---|
| 1 | TPX2 |
| 2 | FOXM1 |
| 3 | CDC2 |
| 4 | MCM2 |
| 5 | H2AFZ |
| 6 | TOP2A |
| 7 | PCNA |
| 8 | UBE2C |
| 9 | MELK |
| 10 | TRIP13 |
| 11 | MCM7 |
| 12 | CDC45L |
| 13 | MAD2L1 |
| 14 | ESPL1 |
| 15 | CCNB2 |
| 16 | FEN1 |
| 17 | TTK |
| 18 | CCT5 |
| 19 | RFC4 |
| 20 | ch-TOG |
| 21 | NUP205 |
| 22 | CDC20 |
| 23 | CKS2 |
| 24 | CCNB1 |
| 25 | AURKB |
| 26 | MSH6 |
| 27 | EZH2 |
| 28 | OIP5 |
| 29 | PTTG1 |
| 30 | H2AFX |
| 31 | ZWINT |
| 32 | CDC6 |
| 33 | UNG |
| 34 | RAD21 |
| 35 | ACTL6A |
| 36 | DHCR7 |
| 37 | STK6 |
| 38 | KIAA0286 |
| 39 | SNRPB/MGC10715 |
| 40 | TK1 |
| 41 | HNRPAB |
| 42 | MKI67 |
| 43 | CENPF |
| 44 | Spc25 |
| 45 | DLG7 |
| 46 | HPRT1 |
| 47 | KNTC2 |
| 48 | MSH2 |
| 49 | NUP155 |
| 50 | POP7 |
| 51 | LMNB1 |
| 52 | CDKN3 |
| 53 | LRP8 |
| 54 | TYMS |
| 55 | CCNA2 |
| 56 | MTHFD2 |
| 57 | RFC2 |
| 58 | MCM6 |
| 59 | FANCG |
| 60 | MYBL2 |
| 61 | MCM3 |
| 62 | NCOA6 |
| 63 | EIF2C2 |
| 64 | TROAP |
| 65 | SIL |
| 66 | PRIM1 |
| 67 | POLD2 |
| 68 | EST1B |
| 69 | GGH |

Data derived from a set of genes can include the expression level measurement of each of the genes in the set or for a subset of genes in a gene set as well as other measurements related to the genes as described herein. The data of the other measurements can be independent of the expression levels. Further, such data can be contained on a computer readable medium.

The foregoing, and other features and advantages of the present teachings, will be more fully understood from the following description and claims.

### DETAILED DESCRIPTION

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

Human solid tumors exhibit differences in outcome even for the same tumor type. Thus, if a clinician can determine which outcome is probable for a specific tumor, the clinician would know if a more or less aggressive treatment regimen can be used. One possibility is to consider the amount of chromosomal aberrations that exists in the specific tumor. It is known for those trained in the art that there is a strong correlation between the total number of chromosomal aberrations in a given tumor and its malignancy. High numbers of chromosomal aberrations are usually associated with a more malignant phenotype.

With respect to the chromosomal complement of a solid human tumor, the tumor exhibits various aberrations such as multiple trisomies, tetrasomy, and multiple translocations and deletions. These aberrations in chromosomal stability are found in solid tumors of the lung, prostate, breast, brain (both medulloblastoma and glioma), and lymph nodes (lymphoma). To quantify the amount of chromosomal aberrations, one may apply any of the following three methods: 1) counting the number of morphologically visible chromosomal aberrations by the application chromosome visualization methods such as spectral karyotyping; 2) quantifying the amount of chromosomal aberrations obtained by array comparative genomic hybridization (aCGH); and 3) quantifying chromosomal aberrations by their effect on the expression level of the genes contained in a given chromosomal region. The latter method, which is an integral part of the present teachings, produces a measure called “functional aneuploidy.”

The numerical and structural chromosomal aberrations seen in malignancies are a consequence of the aberrant functioning of the cell's mechanism to maintain genomic integrity. This cellular aberration is called “chromosomal instability” (CIN). Similarly to aneuploidy, its causative mechanism, CIN is also associated with malignancies. High levels of CIN are expected to confer a more malignant phenotype. Despite the obvious utility of quantifying CIN for clinical diagnostics, its application has been hindered by technical difficulties. The current patent application provides a readily applicable quantification method of CIN in clinical tumor samples.

A gene expression signature of CIN is derived by the identification of genes with the highest level of correlation between a gene's expression level and the overall level of chromosomal aberrations across a given set of cancer samples.

The overall level of chromosomal aberrations in a given clinical sample can be derived by any of the three techniques described herein.

In cancer cells chromosomes can be visualized by spectral karyotyping (SKY) that allows counting the total number of chromosomes and morphological aberrations of chromosomes such as deletions, insertions, translocations, and inversions of various chromosomal regions. In one embodiment the total number of such numerical and morphological aberrations in a cancer cell is used to estimate the overall level of chromosomal aberrations.

In cancer cells the copy number of each chromosomal region can be measured by array comparative genomic hybridization using microarrays by containing either long cDNA clones targeting the individual chromosomal regions or short DNA probes, such as those used on the so-called single nucleotide polymorphism (SNP) chips. In one embodiment the total number of chromosomal aberrations in a cancer sample is calculated by adding up the deviation of each chromosomal region from the normal chromosomal copy number across the entire genome.

In cancer cells chromosomal copy number changes have a direct impact on the RNA expression level of the genes contained in a given chromosomal region. Therefore, chromosomal copy number changes can be estimated by calculating the net deviation of the expression level of all genes contained in a given chromosomal region relative to the remainder of the sampled transcriptome.

First a tumor sample from each of the solid tumors of interest was obtained. A microarray was then used to quantify the expression level of a large number, typically 10,000-20,000 genes in each tumor sample. For a given microarray, each probe or probe set was first mapped to its corresponding transcriptome by sequence mapping and then, through this transcript, the microarray probes were mapped to their respective chromosomal cytobands.

For each chromosomal cytoband, all of the genes present in the microarray measurement that map to that region are grouped into a set designated B (short for band). In one embodiment, if less than ten genes were mapped to a band, the group was disregarded as statistically unreliable. Although in this embodiment the mapping of genes to the cytobands of the chromosome was used to group the genes, it is contemplated that the grouping of genes into statistically meaningful sets can be accomplished by using windows of equal linear length along the chromosome (5-30 Mb long) or genes can be grouped by neighborhood criteria (20 to 100 genes that are located next to each other on the same chromosome would form a set of genes for further analysis). Also, although ten genes were considered the minimum number of genes necessary to form a group, it is contemplated that other numbers of genes can be used to determine statistical reliability.

The rest of the genes, i.e. the rest of the transcriptome that is localized somewhere else on the chromosomes and which are measured on the same microarray, are grouped into a set G (short for genome). The sets B and G are disjoint. The distributions of the genes in B and G are then compared using an appropriate statistical metric, such as the t-statistic. In one embodiment, the statistical significance of the group of genes was determined by taking the mean of the log to the base ten of the expression level of each gene in the group B and comparing it with the expression level of the genes from group G. In general, the statistical metric is formed on a linear combination of the expression level of the genes in the set of genes. The expression levels can be weighted. Other statistical tests, which can be used include: Wilcoxon-Rank test, Signal to Noise ratio, Kolmogorov-Smirnov test and Kruskal-Wallis test

This process is iterated for each gene expression profile in a given cohort such that upon termination, a matrix of t-statistics for each of approximately 350 cytobands per hybridization was obtained. The thus created statistical measures will provide an estimate of the level of aberrant gene expression of a given gene set contained within a given chromosomal region. This is a basis of functional aneuploidy, a measure of the impact of chromosomal aberrations on the transcriptome.

In addition to the measures outlined above, the overall level of chromosomal aberrations can be characterized by summing up the level of functional aneuploidy across all chromosomal regions. This novel measure is termed total functional aneuploidy.

For a given set of cancer samples the following measures are obtained: (a) gene expression measurements at the RNA level for typically 10,000-20,000 genes, usually but not exclusively obtained by microarray measurements. This is a key for all subsequent steps. In addition to this the following measures may also be obtained (b) array comparative genomic hybridization across the entire genome and/or (c) a detailed morphological characterization of all chromosomal aberrations.

For each gene in a given cancer data the gene's expression level across all samples will form a gene expression vector. The total number of chromosomal aberrations in the individual cancer samples as determined by the total number of morphological aberrations, total number of aCGH based chromosomal copy number deviations and total functional aneuploidy will form three additional vectors. Correlation between each gene expression vector and the three vectors characterizing the overall level of chromosomal aberrations is calculated for all genes. The genes with the highest level of correlation to the overall level of chromosomal aberrations will form the CIN gene expression signature.

A group of expressed genes in a tumor which was difficult to treat showed increased expression relative to tumors which were easier to treat. These genes included:

| | |
|---|---|
| 1 | TPX2 |
| 2 | PRC1 |
| 3 | FOXM1 |
| 4 | CDC2 |
| 5 | C20 or f24/TGIF2 |
| 6 | MCM2 |
| 7 | H2AFZ |
| 8 | TOP2A |
| 9 | PCNA |
| 10 | UBE2C |
| 11 | MELK |
| 12 | TRIP13 |
| 13 | CNAP1 |
| 14 | MCM7 |
| 15 | RNASEH2A |
| 16 | RAD51AP1 |
| 17 | KIF20A |
| 18 | CDC45L |
| 19 | MAD2L1 |
| 20 | ESPL1 |
| 21 | CCNB2 |
| 22 | FEN1 |
| 23 | TTK |
| 24 | CCT5 |
| 25 | RFC4 |
| 26 | ATAD2 |
| 27 | ch-TOG |
| 28 | NUP205 |
| 29 | CDC20 |
| 30 | CKS2 |
| 31 | RRM2 |
| 32 | ELAVL1 |
| 33 | CCNB1 |
| 34 | RRM1 |
| 35 | AURKB |
| 36 | MSH6 |
| 37 | EZH2 |
| 38 | CTPS |
| 39 | DKC1 |
| 40 | OIP5 |
| 41 | CDCA8 |
| 42 | PTTG1 |
| 43 | C10orf3 |
| 44 | H2AFX |
| 45 | CMAS |
| 46 | BRRN1 |
| 47 | MCM10 |
| 48 | LSM4 |
| 49 | MTB |
| 50 | ASF1B |
| 51 | ZWINT |
| 52 | TOPK |
| 53 | FLJ10036 |
| 54 | CDCA3 |
| 55 | ECT2 |
| 56 | CDC6 |
| 57 | UNG |
| 58 | MTCH2 |
| 59 | RAD21 |
| 60 | ACTL6A |
| 61 | GPI and MGC13096 |
| 62 | SFRS2 |
| 63 | HDGF |
| 64 | NXT1 |
| 65 | NEK2 |
| 66 | DHCR7 |
| 67 | STK6 |
| 68 | NDUFAB1 |
| 69 | KIAA0286 |
| 70 | KIF4A |
| 71 | SNRPB/MGC10715 |
| 72 | UCK2 |
| 73 | PARP1 |
| 74 | RAD54L |
| 75 | NUSAP1 |
| 76 | RFC5 |
| 77 | TK1 |
| 78 | WBP11 |
| 79 | SYNCRIP/SNX14 |
| 80 | BIRC5 and AFMID |
| 81 | HNRPAB |
| 82 | TACC3 |
| 83 | MKI67 |
| 84 | CENPF |
| 85 | Spc25 |
| 86 | C20orf172 |
| 87 | PTBP1 |
| 88 | DLG7 |
| 89 | POLR2K |
| 90 | IARS |
| 91 | HPRT1 |
| 92 | NSDHL |
| 93 | KNTC2 |
| 94 | RAMP |
| 95 | C10orf7 |
| 96 | C12orf14 |
| 97 | SNRPD1 |
| 98 | FLJ20989 |
| 99 | NIF3L1 |
| 100 | DER1 |

Many of these genes are known to be related to chromosomal stability and hence are consistent with chromosomal aberrations as a cause of the malignant phenotype. The application of the method to multiple datasets indicates that the following genes consistently have increased expression in difficult-to-treat tumors:

| | |
|---|---|
| 1 | TPX2 |
| 2 | FOXM1 |
| 3 | CDC2 |
| 4 | MCM2 |
| 5 | H2AFZ |
| 6 | TOP2A |
| 7 | PCNA |
| 8 | UBE2C |
| 9 | MELK |
| 10 | TRIP13 |
| 11 | MCM7 |
| 12 | CDC45L |
| 13 | MAD2L1 |
| 14 | ESPL1 |
| 15 | CCNB2 |
| 16 | FEN1 |
| 17 | TTK |
| 18 | CCT5 |
| 19 | RFC4 |
| 20 | ch-TOG |
| 21 | NUP205 |
| 22 | CDC20 |
| 23 | CKS2 |
| 24 | CCNB1 |
| 25 | AURKB |
| 26 | MSH6 |
| 27 | EZH2 |
| 28 | OIP5 |
| 29 | PTTG1 |
| 30 | H2AFX |
| 31 | ZWINT |
| 32 | CDC6 |
| 33 | UNG |
| 34 | RAD21 |
| 35 | ACTL6A |
| 36 | DHCR7 |
| 37 | STK6 |
| 38 | KIAA0286 |
| 39 | SNRPB and |
| | MGC10715 |
| 40 | TK1 |
| 41 | HNRPAB |
| 42 | MKI67 |
| 43 | CENPF |
| 44 | Spc25 |
| 45 | DLG7 |
| 46 | HPRT1 |
| 47 | KNTC2 |
| 48 | MSH2 |
| 49 | NUP155 |
| 50 | POP7 |
| 51 | LMNB1 |
| 52 | CDKN3 |
| 53 | LRP8 |
| 54 | TYMS |
| 55 | CCNA2 |
| 56 | MTHFD2 |
| 57 | RFC2 |
| 58 | MCM6 |
| 59 | FANCG |
| 60 | MYBL2 |
| 61 | MCM3 |
| 62 | NCOA6 |
| 63 | EIF2C2 |
| 64 | TROAP |
| 65 | SIL |
| 66 | PRIM1 |
| 67 | POLD2 |
| 68 | EST1B |
| 69 | GGH |

Therefore, by determining that these sets of tumor genes or an appropriate subset thereof have an elevated expression level, the clinician can determine that the tumor is difficult to treat.

The present teachings encompass embodiments in other specific forms without departing from the spirit or essential characteristics thereof. The foregoing embodiments are therefore to be considered in all respects illustrative rather than limiting on the present teachings described herein. Scope of the present invention is thus indicated by the appended claims rather than by the foregoing description, and all changes that come within the meaning and range of equivalency of the claims are intended to be embraced therein.

1. A method of predicting an outcome of the treatment of a human solid tumor, the method comprising:
measuring in a tumor cell the mRNA expression of at least 25 genes in the following set of genes:

| | |
|---|---|
| 1 | TPX2 |
| 2 | PRC1 |
| 3 | FOXM1 |
| 4 | CDC2 |
| 5 | C20 or f24/TGIF2 |
| 6 | MCM2 |
| 7 | H2AFZ |
| 8 | TOP2A |
| 9 | PCNA |
| 10 | UBE2C |
| 11 | MELK |
| 12 | TRIP13 |
| 13 | CNAP1 |
| 14 | MCM7 |
| 15 | RNASEH2A |
| 16 | RAD51AP1 |
| 17 | KIF20A |
| 18 | CDC45L |
| 19 | MAD2L1 |
| 20 | ESPL1 |
| 21 | CCNB2 |
| 22 | FEN1 |
| 23 | TTK |
| 24 | CCT5 |
| 25 | RFC4 |
| 26 | ATAD2 |
| 27 | ch-TOG |
| 28 | NUP205 |
| 29 | CDC20 |
| 30 | CKS2 |
| 31 | RRM2 |
| 32 | ELAVL1 |
| 33 | CCNB1 |
| 34 | RRM1 |
| 35 | AURKB |
| 36 | MSH6 |
| 37 | EZH2 |
| 38 | CTPS |
| 39 | DKC1 |
| 40 | OIP5 |
| 41 | CDCA8 |
| 42 | PTTG1 |
| 43 | C10 or f3 |
| 44 | H2AFX |
| 45 | CMAS |
| 46 | BRRN1 |
| 47 | MCM10 |
| 48 | LSM4 |
| 49 | MTB |
| 50 | ASF1B |
| 51 | ZWINT |
| 52 | TOPK |
| 53 | FLJ10036 |
| 54 | CDCA3 |
| 55 | ECT2 |
| 56 | CDC6 |
| 57 | UNG |
| 58 | MTCH2 |
| 59 | RAD21 |
| 60 | ACTL6A |
| 61 | GPI and MGC13096 |
| 62 | SFRS2 |
| 63 | HDGF |
| 64 | NXT1 |
| 65 | NEK2 |
| 66 | DHCR7 |
| 67 | STK6 |
| 68 | NDUFAB1 |
| 69 | KIAA0286 |
| 70 | KIF4A |
| 71 | SNRPB/MGC10715 |
| 72 | UCK2 |
| 73 | PARP1 |
| 74 | RAD54L |
| 75 | NUSAP1 |
| 76 | RFC5 |
| 77 | TK1 |
| 78 | WBP11 |
| 79 | SYNCRIP/SNX14 |
| 80 | BIRC5 and AFMID |
| 81 | HNRPAB |
| 82 | TACC3 |
| 83 | MKI67 |
| 84 | CENPF |
| 85 | Spc25 |
| 86 | C20orf172 |
| 87 | PTBP1 |
| 88 | DLG7 |
| 89 | POLR2K |
| 90 | IARS |
| 91 | HPRT1 |
| 92 | NSDHL |
| 93 | KNTC2 |
| 94 | RAMP |
| 95 | C10 or f7 |
| 96 | C12 or f14 |
| 97 | SNRPD1 |
| 98 | FLJ20989 |
| 99 | NIF3L1 |
| 100 | DER1 |

taking a statistical measure of the expression level of the measured genes; and
if the statistical measure of the expression level of the measured genes is elevated, determining to a 99% confidence level that the prognosis is poor.

2. The method of claim 1 wherein the solid tumor is of a cancer selected from lung cancer, prostate cancer, medulloblastoma, glioma, breast cancer and lymphoma.

3. The method of claim 1 wherein the statistical measure of the expression level of the measured genes is a linear combination of the expression level of the genes in the set of genes.

4. The method of claim 3 wherein the linear combination of the expression level of the genes in the set of genes is a combination of weighted expression levels.

5. The method of claim 3 wherein the linear combination of the expression level of the genes in the set of genes is the mean of logarithms of the expression levels.

6. The method of claim 1 wherein the statistical measure of the expression level of the measured genes is elevated relative to the expression level of the measured genes from a tumor whose prognosis is good.

7. The method of claim 1 further comprising taking a biopsy of a human solid tumor.

8. The method of claim 1 wherein the measuring in the tumor cells the RNA expression comprises using a microarray.

9. A method of predicting an outcome of the treatment of a human solid tumor, the method comprising:
measuring in the cells of a tumor the expression level of a set of genes whose change is related to chromosomal instability;
taking a statistical measure of the expression level of the set of measured genes; and
if the statistical measure of the expression level of the set of measured genes is elevated, determining that the prognosis is poor.

10-14. (canceled)

15. A set of genes useful in determining the outcome of treatment of solid tumors, the set of genes consisting essentially of:

| | |
|---|---|
| 1 | TPX2 |
| 2 | PRC1 |
| 3 | FOXM1 |
| 4 | CDC2 |
| 5 | C20orf24andTGIF2 |
| 6 | MCM2 |
| 7 | H2AFZ |
| 8 | TOP2A |
| 9 | PCNA |
| 10 | UBE2C |
| 11 | MELK |
| 12 | TRIP13 |
| 13 | CNAP1 |
| 14 | MCM7 |
| 15 | RNASEH2A |
| 16 | RAD51AP1 |
| 17 | KIF20A |
| 18 | CDC45L |
| 19 | MAD2L1 |
| 20 | ESPL1 |
| 21 | CCNB2 |
| 22 | FEN1 |
| 23 | TTK |
| 24 | CCT5 |
| 25 | RFC4 |
| 26 | ATAD2 |
| 27 | ch-TOG |
| 28 | NUP205 |
| 29 | CDC20 |
| 30 | CKS2 |
| 31 | RRM2 |
| 32 | ELAVL1 |
| 33 | CCNB1 |
| 34 | RRM1 |
| 35 | AURKB |
| 36 | MSH6 |
| 37 | EZH2 |
| 38 | CTPS |
| 39 | DKC1 |
| 40 | OIP5 |
| 41 | CDCA8 |
| 42 | PTTG1 |
| 43 | C10orf3 |
| 44 | H2AFX |
| 45 | CMAS |
| 46 | BRRN1 |
| 47 | MCM10 |
| 48 | LSM4 |
| 49 | MTB |
| 50 | ASF1B |
| 51 | ZWINT |
| 52 | TOPK |
| 53 | FLJ10036 |
| 54 | CDCA3 |
| 55 | ECT2 |
| 56 | CDC6 |
| 57 | UNG |
| 58 | MTCH2 |
| 59 | RAD21 |
| 60 | ACTL6A |
| 61 | GPIandMGC13096 |
| 62 | SFRS2 |
| 63 | HDGF |
| 64 | NXT1 |
| 65 | NEK2 |
| 66 | DHCR7 |
| 67 | STK6 |
| 68 | NDUFAB1 |
| 69 | KIAA0286 |
| 70 | KIF4A |
| 71 | SNRPBandMGC10715 |
| 72 | UCK2 |
| 73 | PARP1 |
| 74 | RAD54L |
| 75 | NUSAP1 |
| 76 | RFC5 |
| 77 | TK1 |
| 78 | WBP11 |
| 79 | SYNCRIPandSNX14 |
| 80 | BIRC5andAFMID |
| 81 | HNRPAB |
| 82 | TACC3 |
| 83 | MKI67 |
| 84 | CENPF |
| 85 | Spc25 |
| 86 | C20orf172 |
| 87 | PTBP1 |
| 88 | DLG7 |
| 89 | POLR2K |
| 90 | IARS |
| 91 | HPRT1 |
| 92 | NSDHL |
| 93 | KNTC2 |
| 94 | RAMP |
| 95 | C10orf7 |
| 96 | C12orf14 |
| 97 | SNRPD1 |
| 98 | FLJ20989 |
| 99 | NIF3L1 |
| 100 | DER1 |

16-18. (canceled)

19. A data set comprising the expression levels measured from a human solid tumor, wherein the expression levels are of the set of genes of claim 15.

20. The data set of claim 19 on a computer-readable medium.

21. The data set of claim 19 displayed on a computer screen or visualized on a tangible medium.
